(19) United States
(12) Patent Application Publication
(10) Pub. No.: US 2021/0104314 A1
(43) Pub. Date: Apr. 8, 2021

(54) MOBILE DEVICE CONFIGURATION FOR TRACKING INSTALLATION AND DISPOSAL OF A MEDICAL LINEN

(71) Applicant: On the Right Track Systems, Inc., New York, NY (US)

(72) Inventor: Ajay Singhal, Jericho, NY (US)

(73) Assignee: On the Right Track Systems, Inc., New York, NY (US)

(21) Appl. No.: 16/592,699

(22) Filed: Oct. 3, 2019

(51) Int. Cl. G16H 40/20; G16H 40/40; G06K 7/14

(52) U.S. Cl. CPC G16H 40/20; G06K 7/1443; G06K 7/1417; G16H 40/40

(57) ABSTRACT

A mobile computing device has an image capture device configured to scan a quick response code adhered to a medical linen. Further, the mobile computing device has a processor that generates a graphical user interface having a scanning menu, which lists one or more user selectable indicia. Each of the one or more user selectable indicia is selectable prior to the image capture device performing the scan of the quick response code. Furthermore, each of the one or more selectable indicia is associated with a computer-implemented instruction to update a database via a network. The database stores a data structure corresponding to the medical linen. The one or more selectable indicia are selected from the group consisting of: a medical linen installation indicium and a medical linen disposal indicium.

### BACKGROUND

### 1. Field

This disclosure generally relates to a medical environment. More particularly, the disclosure relates to the field of medical linens within a medical environment.

### 2. General Background

Medical environments (e.g., hospitals, doctor's offices, etc.) are typically prone to the spread of germs, bacteria, and mold. For example, the linens (e.g., curtains, sheets, pillow cases, blankets, etc.) used within a hospital environment may be susceptible to methicillin-resistant Staphylococcus aureus (“MRSA”), which is a dangerous bacteria. Because of a significant amount of touching from multiple patients, curtains that separate one patient from another tend to be prone to gathering MRSA within only a short period of time. As another example, linens may be susceptible to Clostridium difficile (“C. difficile”), which is another bacteria that is harmful.

Although the foregoing health risks are quite significant, many medical environments rely on inefficient approaches that do not adequately ensure the cleanliness of linens. For example, some medical environments will wait until a hospital curtain appears to be visibly dirty before attempting to clean or change it—in the meantime MRSA and other contaminants may have easily accumulated to dangerous levels within that hospital curtain. As another example, some medical environments attempt to use a log book for the laundering and changing of medical linens, but such an approach is often haphazard because the log book is typically based on a quantity of linens, without any particularity. As a result, the linens in a closet in one hospital room may not make it into the laundering process, whereas linens from another room may get laundered. Ultimately, medical staff within a medical environment typically does not know when medical linens were actually laundered.

Given the importance of cleanliness within a medical environment, current approaches to laundering and changing medical linens are inadequate for avoiding significant health risks.

### SUMMARY

A mobile computing device has an image capture device configured to scan a quick response (“QR”) code adhered to a medical linen. Further, the mobile computing device has a processor that generates a graphical user interface (“GUI”) having a scanning menu, which lists one or more user selectable indicia. Each of the one or more user selectable indicia is selectable prior to the image capture device performing the scan of the QR code. Furthermore, each of the one or more selectable indicia is associated with a computer-implemented instruction to update a database via a network. The database stores a data structure corresponding to the medical linen. The one or more selectable indicia are selected from the group consisting of: a medical linen installation indicium and a medical linen disposal indicium. Additionally, the mobile computing device has a display device that displays the GUI and the scanning menu within the GUI. Further, the mobile computing device has a receiver that receives a user input, via the GUI, of a selected indicium from the one or more user selectable indicia. The mobile computing device also has a memory device that stores data corresponding to the selected indicium. The memory device further stores the image of the QR code subsequent to storing the selected indicium. Finally, the mobile computing device has a transmitter that sends the selected indicium and the QR code to the server.

As an alternative, a computer program may have a computer readable storage device with a computer readable program stored thereon that implements the functionality of the aforementioned mobile computing device. As yet another alternative, a process that utilizes a processor may implement the functionality of the aforementioned mobile computing device.

As another alternative, a system may have a database that stores a data structure corresponding to a medical linen. Furthermore, the system may have a processor that generates a GUI having a scanning menu, which lists one or more user selectable indicia. Each of the one or more user selectable indicia is selectable prior to an image capture device of a mobile computing device performing a scan of a QR code adhered to a medical linen. Further, each of the one or more selectable indicia is associated with a computer-implemented instruction to update the database via a network. The one or more selectable indicia are selected from the group consisting of: a medical linen installation indicium, a laundered medical linen indicium, and a medical linen disposal indicium. Finally, the system may have a transmitter that sends the GUI to the mobile computing device prior to a user selection being performed from the scanning menu.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1 illustrates a computerized medical linen tracking platform.

FIG. 2 illustrates a system configuration for the server and/or the mobile computing device illustrated in FIG. 1.

FIG. 3A illustrates a display screen of the mobile computing device rendering a GUI for a menu of application functions provided by an application executed by the mobile computing device.

FIG. 3B illustrates the display screen of the mobile computing device rendering the GUI.

FIG. 3C illustrates the application being configured to activate a QR code scanning window.

FIG. 3D illustrates an example of the GUI displaying a medical linen history for a particular medical linen.

FIG. 3E illustrates a geolocation map that may be rendered by the GUI.

FIG. 4A illustrates an example of a medical environment with a medical linen that is a medical curtain separating hospital beds, each having a patient situated thereon, respectively.

FIG. 4B illustrates an example of the user scanning the QR code after the medical curtain has been laundered (i.e., washed, dried, etc.) by a laundry machine.

FIG. 4C illustrates the user scanning the QR code as the medical curtain is about to be disposed of in a trash container.

FIG. 5 illustrates a process that may be utilized by the mobile computing device to generate the GUI illustrated in FIGS. 3A-3E.

### DETAILED DESCRIPTION

A mobile device configuration is provided to track the laundering of textile-based medical linens (e.g., curtains, sheets, and pillow covers made from cloth). The mobile device configuration allows a user (e.g., hospital worker) to effectively input various actions pertaining to the laundering of the textile-based medical linens based upon one or more user inputs to a mobile computing device (e.g., smartphone, tablet device, smartwatch, smart wearable, etc.). In essence, the mobile device configuration improves the usability of a mobile computing device via a GUI to allow the user to effectively manage laundering actions performed with respect to textile-based medical linens. Further, the mobile device configuration allows a user to periodically monitor the status of the various textile-based medical linens via the GUI of the mobile computing device, thereby leading to improved management of textile-based medical linens within a medical environment. Further, a user may scan the QR code of the textile-based medical linen to determine the laundering history of the textile-based medical linen. Finally, the user may use the GUI to view a graphical representation, two-dimensional (“2-D”) or three-dimensional (“3-D”), of a medical environment in which the curtains are positioned, and their relative positioning within that medical environment. Therefore, the mobile device configuration improves the usability of a mobile computing device to allow for laundering event management, laundering event tracking, and visual depiction of the textile-based medical linens and corresponding statuses within the medical environment.

With the foregoing improved usability of the mobile computing device, hospital workers and/or vendors are able to effectively sanitize the medical environment. Whether a hospital worker is present within a particular hospital room determining the laundering history of a particular medical linen (e.g., a medical curtain separating hospital room beds) or in a hospital hallway trying to get an overview of multiple medical curtain laundering statuses, the hospital worker is provided with a mobile configuration that allows the hospital worker to go from room to room to maintain the cleanliness of the hospital environment, thereby reducing the risk of medical linens, and ultimately patients, being contaminated with MRSA, C. difficile, or other health hazards.

Alternatively, the mobile device configuration may be used to track the installation/disposal of disposable medical linens. A similar GUI may be used to that of textile-based medical linens, with the exception of features associated with the laundering of a medical linen.

Furthermore, the GUI in any of the configurations provided for herein may be configured to automatically display one or more alerts. For example, a time threshold parameter may be configured by a processor such that a medical linen being installed for a time period that exceeds that threshold parameter triggers an alert to replace the medical linen, whereby the alert is displayed via the GUI.

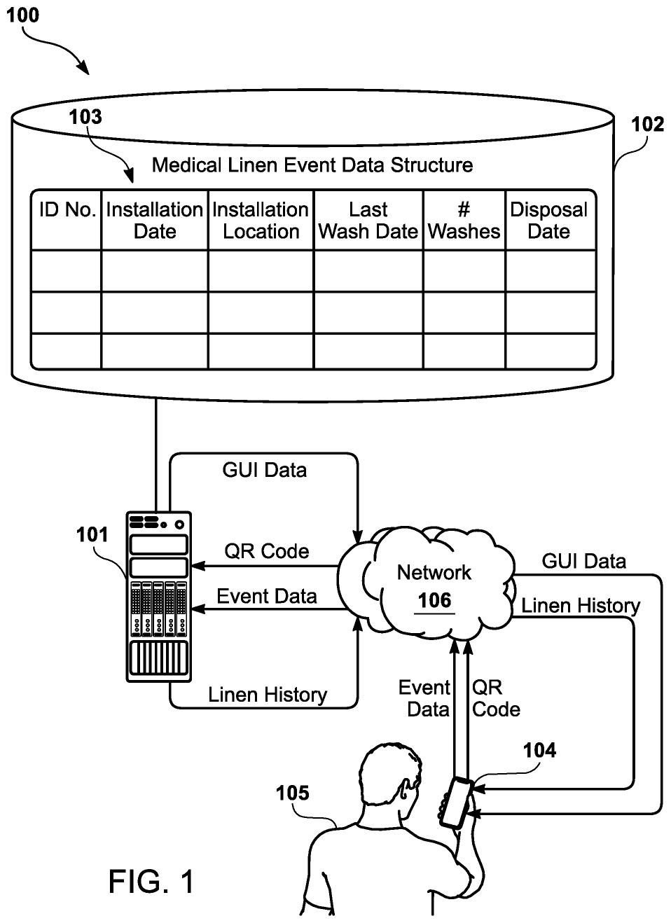

FIG. 1 illustrates a computerized medical linen tracking platform 100. In particular, the computerized medical linen tracking platform 100 has a server 101 that is configured to provide functionality to an application via a mobile computing device 104 operated by a user 105. In other words, the computerized medical linen tracking platform 100 may be a cloud-based solution in which the server 101 communicates with the mobile computing device 104 to provide the medical linen tracking functionality.

As an example, the server 101 may generate a GUI specific to the application to be used by the mobile computing device 104 to perform medical linen tracking. Accordingly, the sever 101 may provide the GUI to the mobile computing device 104, which may execute an application that renders the GUI, based on the GUI data, on a display device of the mobile computing device 104.

The application allows the mobile computing device 104 to receive user inputs to provide data pertaining to a particular medical linen to the server 101. For example, a medical linen may have a QR code adhered thereto, and may be scanned by an image capture device (e.g., camera or other imaging device) integrated within, or in operable communication with, the mobile computing device 104. The QR code may have encoded therein a medical linen identifier. In one embodiment, the application decodes the QR code after performing the scan of the QR code, and sends the decoded medical linen identifier to the server 101. In another embodiment, the application sends the QR code to the server 101, which performs the decoding of the QR code. In addition, the application may send laundering event data along with the QR code (encoded or decoded) to the server 101.

Further, the server 101 may be in operable communication (local or remote) with a database 102, which stores a medical linen event data structure 103. Upon receiving the QR code (encoded or decoded) and determining the medical linen identifier, the server 101 may store the received medical linen event data in the medical linen event data structure 103. As an example, the medical linen event data structure 103 may be a multi-dimensional array, with various rows representing different medical linen identifiers and various columns representing various laundering event data fields. For example, the medical linen event data structure 103 may have the following fields: medical linen identification number, installation date, installation location, last wash date, number of washes, and disposal date. (The fields illustrated in the medical linen event data structure 103 of FIG. 1 are provided only as examples, since other fields may be used instead.) Further, upon a request for a textile-based medical linen laundering event or a disposable medical linen installation/disposal event, the server 101 may retrieve data from the medical linen event data structure 103 to provide that data to the mobile computing device 104.

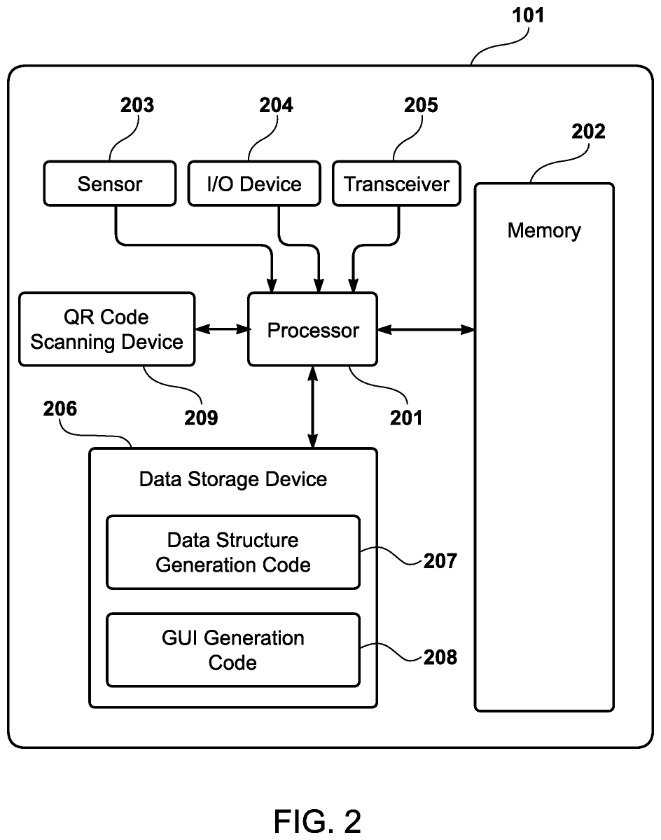

Accordingly, the QR code has dual functionalities for the GUI rendered by the mobile computing device 104: (1) medical linen event inputs for storage in the medical linen event data structure 103; and (2) medical linen event status retrieval from the medical linen event data structure 103. As a result, the GUI enhances usability of the mobile computing device 104 for the user 105 to effectively provide commands to store medical linen events within the medical linen event data structure 103 and obtain medical linen event data, either on a per medical linen basis or for a batch of medical linen records (e.g., multiple rows) from the medical linen event data structure 103. For example, based on the geolocation proximity (e.g., a predetermined distance, a predetermined landmark, a specific floor, etc.) of a user with respect to a number of medical curtains, the application executed by the mobile computing device 104 may be configured to automatically retrieve medical linen event records. Furthermore, the medical linen event data structure 103 may be optimized so that the most geolocation relevant data, at a particular instance in time, within the medical linen event data structure 103 is optimized for accessibility by the server 101 and/or the mobile computing device 104. For example, the mobile computing device 104 may send periodic messages (e.g., at predetermined time intervals) to the server 101 with indications of the geolocation of the user 105. The server 101 may use such indications, at each instance, to query the medical linen event data structure 103 to obtain the medical linen records that are positioned in the closest proximity to the user 105. The server 101 may temporarily store such records in a data cache within a memory device 202 (FIG. 2) or sort the records within the medical linen event data structure 103 to improve search times by the server 101 and/or the mobile computing device 104. Additionally, the server 101 may automatically retrieve a batch of linen records meeting the geolocation proximity for rendering, on the GUI, a visual depiction of the medical environment in which the medical linens are situated.

FIG. 2 illustrates a system configuration for the server 101 and/or the mobile computing device 104 illustrated in FIG. 1. In particular, a processor 201, which may be specialized for generating GUIs and/or data structures may be used to perform the operations illustrated in FIG. 1 for generating a GUI for medical linen laundering event handling to be rendered on the mobile computing device 104 illustrated in FIG. 1. For example, the processor 201 may be capable of generating the interactive graphical components of the GUI and bringing together those interactive graphical components in a manner that improves usage of the mobile computing device 104.

Furthermore, the system configuration may include one or more sensors 203 that are specifically configured to sense data particular to the medical linen event handling. For example, the sensor 203 may be a geolocation-based sensor (e.g., global positioning system (“GPS)) that determines the location of the user 104 with respect to locations of various medical linens as depicted on the GUI of the mobile computing device 104. The sensors 203 may be integrated within a mobile computing device (e.g., smartphone, tablet device, smartwatch, smart bracelet, etc.) used by the user 105, or may be independently operable devices positioned throughout the medical environment. For example, a plurality of cameras and/or motion sensors may be positioned throughout a hospital to determine position of the user 105 relative to a medical linen identified by a QR code so that the server 101 and/or the computing device 104 may determine the relative positioning of the user 105 and/or the medical linens for rendering on the GUI of the mobile computing device 104.

Moreover, the system configuration may have one or more input/output (“I/O”) devices 204 that may receive inputs and provide outputs. Various devices (e.g., keyboard, microphone, mouse, pointing device, hand controller, etc.) may be used for the I/O devices 204.

Additionally, the system configuration may have a QR code scanning device 209, such as an image capture device (e.g., camera, imaging device, etc.). The QR code scanner scans (e.g., captures an image) of the QR code for decoding by the processor 201 to determine a medical linen identifier for data inputted to, or extracted from, the medical linen data structure 103.

The system configuration may also have a transceiver 205 to send and receive data. Alternatively, a separate transmitter and receiver may be used instead.

The system configuration may also include a memory device 202, which may temporarily store computer readable instructions performed by the processor 201. As an example of such computer readable instructions, a data storage device 206 within the system configuration may store data structure generation code 207 and GUI generation code 208. The processor 201 may execute the data structure generation code 207 to generate the medical linen event data structure 103 illustrated in FIG. 1. Furthermore, the processor 201 may execute the GUI generation code 208 to generate the GUI rendered by the application executed by the mobile computing device 104 illustrated in FIG. 1.

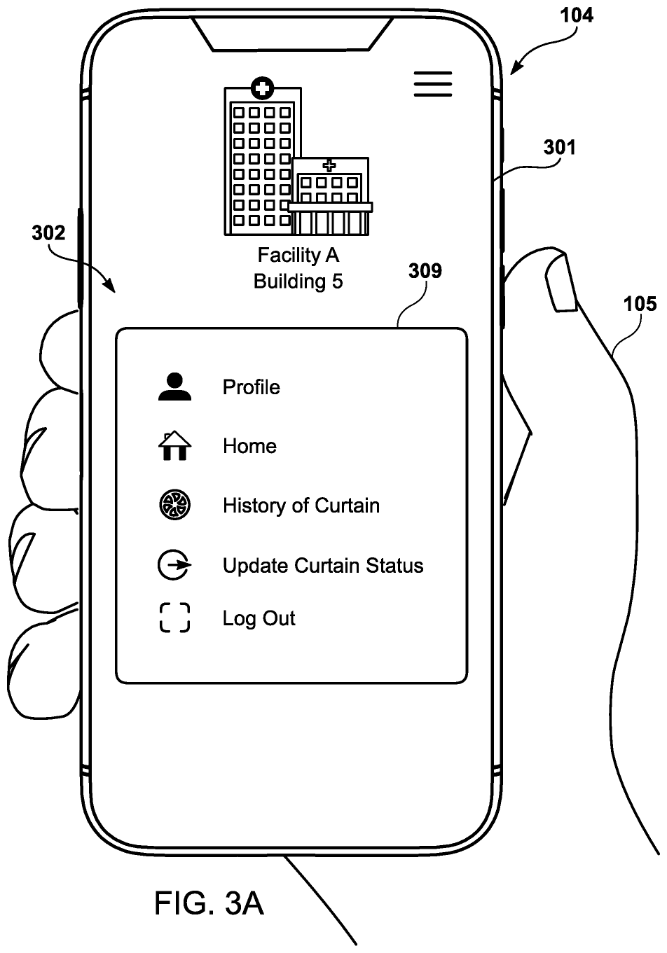

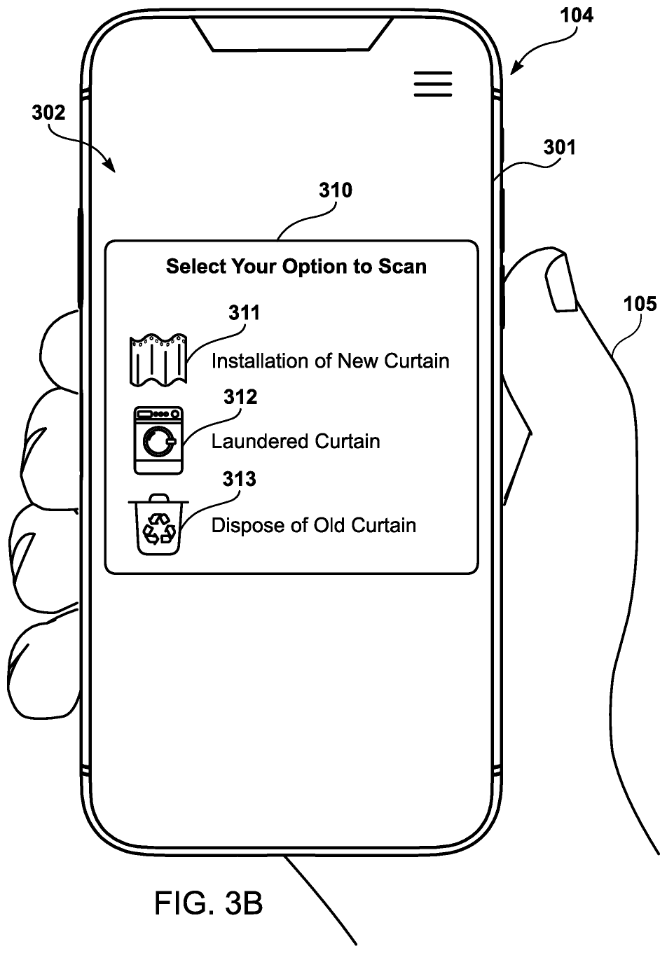

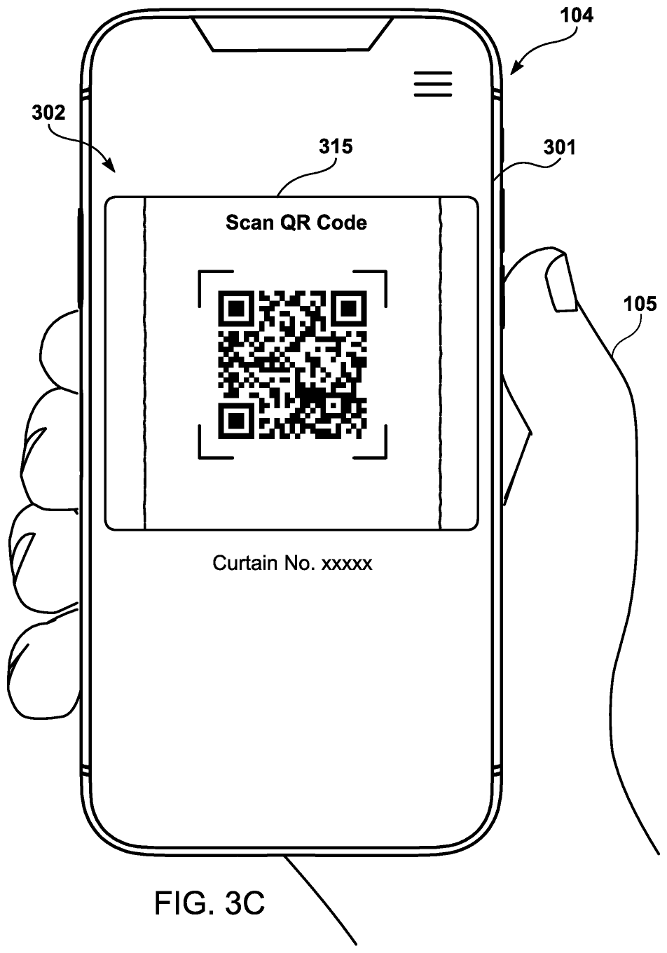

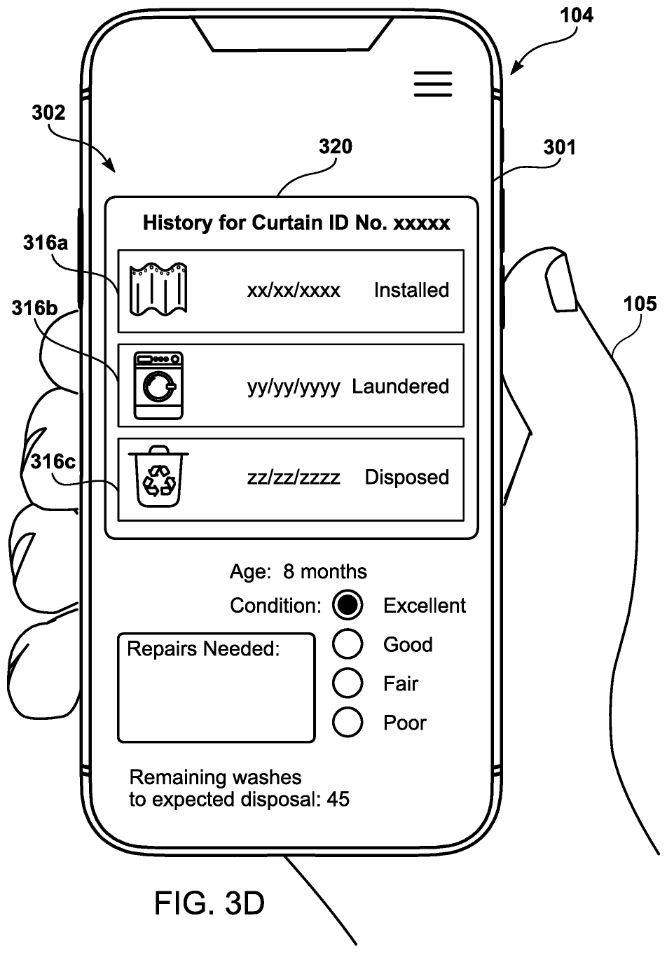

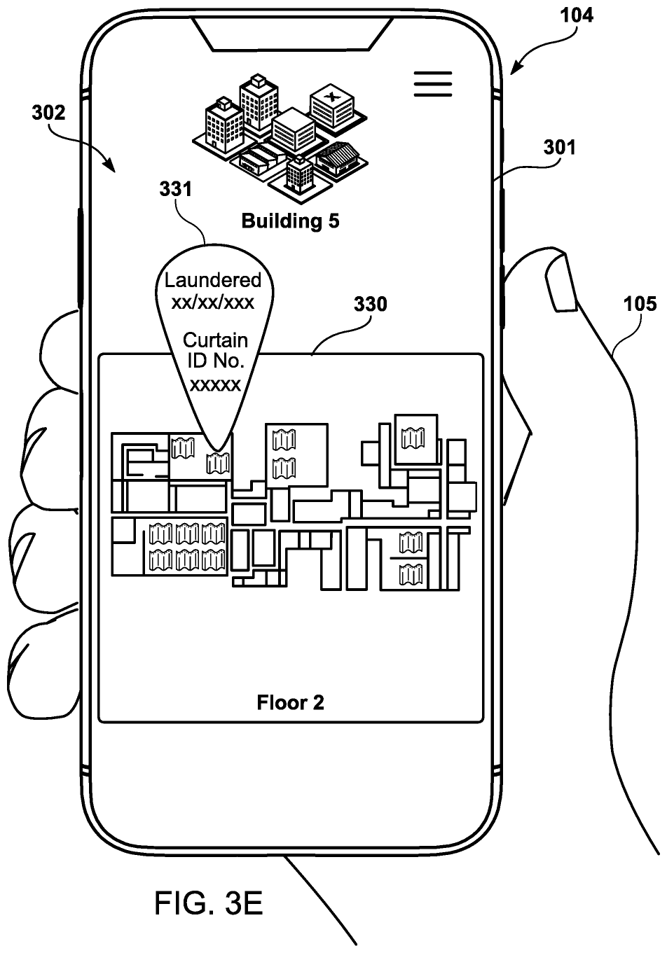

FIGS. 3A-3E illustrate examples of potential displays rendered by the mobile computing device 104 illustrated in FIG. 1. In particular, FIG. 3A illustrates a display screen 301 of the mobile computing device 104 rendering a GUI 302 for a function menu 309 of application functions provided by an application executed by the mobile computing device 104. For example, the GUI 302 may have a menu of various indicia such as a profile indicium, a home indicium, a curtain history indicium, an update curtain status. Accordingly, the user 105 may navigate the application based on this function menu 309. Moreover, the mobile computing device 104 may render an image representing the medical environment (e.g., hospital building) to which the user 105 is relatively geolocated in close proximity.

Furthermore, FIG. 3B illustrates the display screen 301 of the mobile computing device 104 rendering the GUI 302. In particular, the GUI 302 has a scanning menu 310, which lists a plurality of user selectable indicia 311-313. In one embodiment, each of the one or more user selectable indicia may be selectable prior to the QR code scanning device 209 performing the scan of the QR code. In other words, the user 105 may select a command associated with a particular selectable indicium from the scanning menu 310 to determine an action before scanning a QR code. For instance, the command may be one of a plurality of event update commands to update event data stored within the medical linen event data structure 103, whereby each command corresponds to a particular field of the medical linen event data structure 103. For instance, the plurality of user selectable indicia 311-313 may include an installation indicium 311, a laundered curtain indicium 312 (corresponding to a last wash date), and a disposal indicium 313. Subsequent to activating one of the plurality of user selectable indicia 311-313, the application may be configured, via the processor 201 illustrated in FIG. 2, to activate a QR code scanning window 315, as illustrated in FIG. 3C. In an alternative embodiment, the QR code scanning window 315 may allow the user 105 to perform a scan of the QR code prior to selecting one of the plurality of user selectable indicia 311-313.

Conversely, FIG. 3D illustrates an example of the GUI 302 displaying a medical linen history 320 for a particular medical linen. For example, a user 105 may physically enter an area within a medical environment (e.g., a hospital room) and perform a scan of a QR code. The various data fields from the medical linen event data structure 103 may then be retrieved by the server 101 for rendering within the GUI 302 as various history indicia 316a-c so that the user 105 may determine if additional laundering, or other events are necessary, for a particular medical linen.

Additionally, FIG. 3E illustrates a geolocation map 330 that may be rendered by the GUI 302. In one embodiment, the geolocation map 330 displays medical linens according to geolocation proximity (e.g., same floor as the user 105 within a hospital). Further, based on a user input (e.g., a touch-based input), the geolocation map 330 may display various data retrieved from the medical linen data structure 103 in a data indicium 331 (e.g., virtual bubble, pop-up, etc.). For example, the user 105 may take an elevator from the second floor to the tenth floor of a hospital, and find a plurality of medical linen indicia displayed within the geolocation map 330; such fast retrieval and rendering may occur as a result of the processor 201 caching a batch of medical linen records from the medical linen event data structure 103 that are geolocation proximate to the user 105.

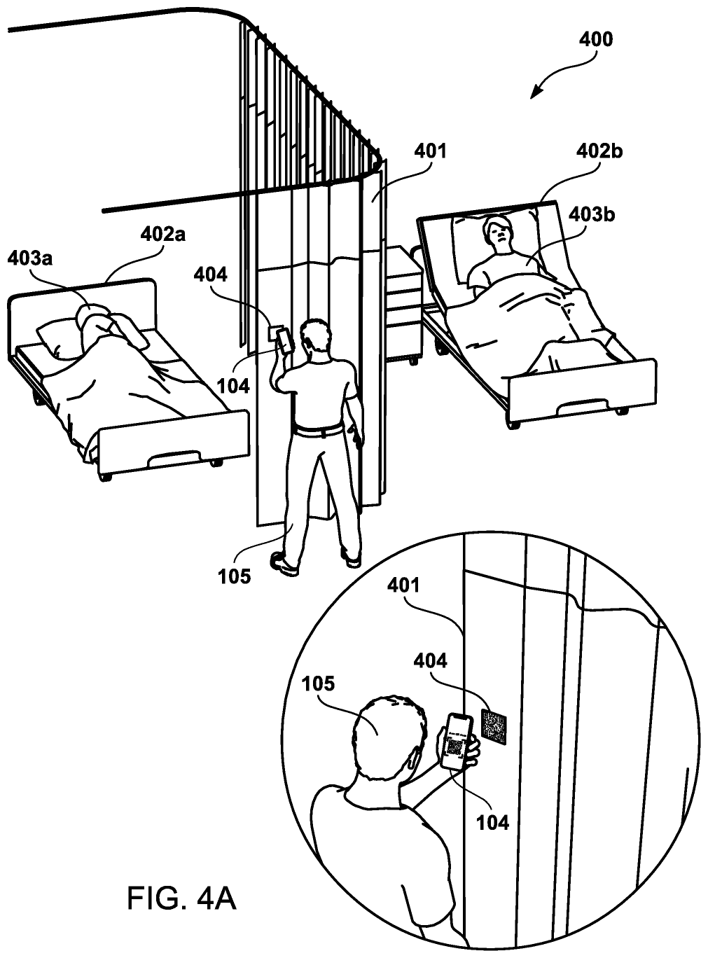

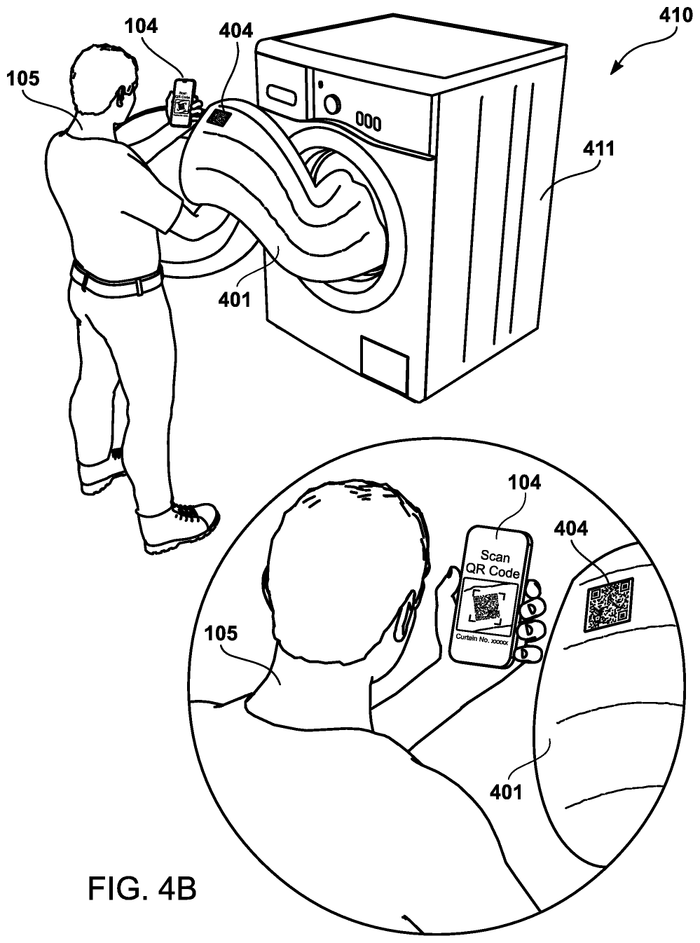

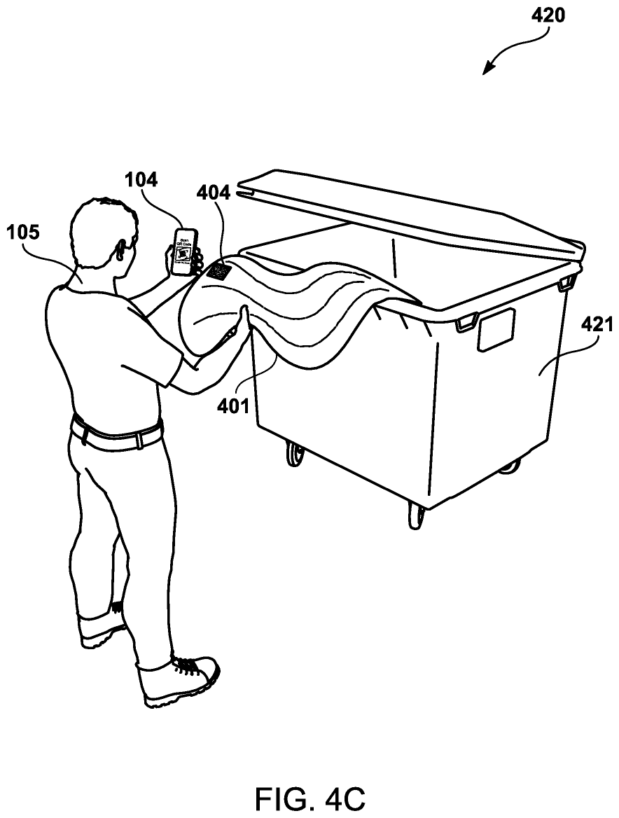

FIGS. 4A-4C illustrate various examples of the user 105 using the mobile computing device 104 to perform medical linen activities based on execution of the application by the mobile computing device 104. For example, FIG. 4A illustrates a medical environment 400 (e.g., hospital room) with a medical linen (e.g., a medical curtain 404) separating hospital beds 402a and 402b, each having patients, 403a and 403b situated thereon, respectively. A QR code 404 is positioned on the medical curtain 401, thereby allowing the user 105 to scan the QR code 404 from the medical curtain 401 to either update the laundering status or obtain the laundering history for that particular medical curtain 401. The QR code 404 may be adhered to (e.g., via stitching, adhesive, embroidered, etc.), or integrated within (e.g., printed), the medical curtain 401.

Additionally, FIG. 4B illustrates an example of a laundering environment 410 in which the user 105 scans the QR code 404 after the medical curtain 401 has been laundered (i.e., washed, dried, etc.) by a laundry machine 411. As yet another example, FIG. 4C illustrates a disposal environment 420 in which the user 105 scans the QR code 404 as the medical curtain 401 is about to be disposed of in a trash container 421. In one embodiment, the medical curtain 401, or other medical linen, is disposable (e.g., a medical substrate such as polypropylene). In another embodiment, the medical curtain 401, or other medical linen, is non-disposable.

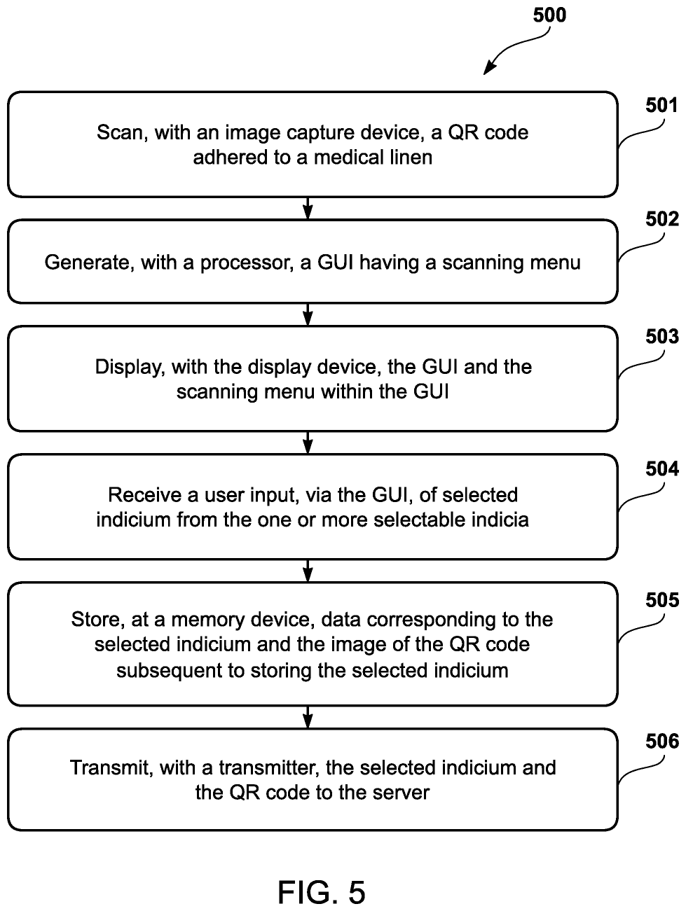

FIG. 5 illustrates a process 500 that may be utilized by the mobile computing device 104 to generate the GUI 302 illustrated in FIGS. 3A-3E. At a process block 501, the process 500 scans, with an image capture device 209, a QR code 404 adhered to a medical curtain 401. Further, at a process block 502, the process 500 generates with the processor 201, illustrated in FIG. 2, the GUI 302 having a scanning menu 310, illustrated in FIG. 3B, which may list one or more user selectable indicia. Each of the one or more user selectable indicia is selectable prior to the image capture device 209 performing the scan of the QR code 404. Further, each of the one or more selectable indicia is associated with a computer-implemented instruction to update the database 102 via the network 106, illustrated in FIG. 1. The database 102 stores the data structure 103 corresponding to the medical linen 401. The one or more selectable indicia is selected from the group consisting of: a medical linen installation indicium, a laundered medical linen indicium, and a medical linen disposal indicium. The process 500 may be used for laundering textile-based medical linens. Alternatively, the process 500 may be used for installation/disposal of disposable medical linens. Various indicia from the plurality of user selectable indicia 311-313 may be displayed and selected based on the type of linen. For example, the GUI 302 may display all of the user selectable indicia 311-313 and history indicia 316a-c for a textile-based medical linen, as illustrated in FIGS. 3B and 3D respectively, or only the installation indicium 311 and the disposal indicium 313, in addition to the installation history indicium 316a and/or the disposal history indicium 316c, for a disposable medical linen. In another embodiment, the GUI 302 has a menu that allows the user 105 to provide a user input indicating the type of medical linen (e.g., textile-based or disposable) for storage in the medical linen event data structure 103, illustrated in FIG. 1. Upon subsequent scanning of the QR code 404, the processor 201 determines the type of the medical linen from the medical linen event data structure 103, and displays a menu with features customized to the type of linen.

Although the QR code 404 is depicted as being scanned by the configurations provided for herein, other encoded, or un-encoded, forms of medical linen identifiers may be used instead.

A computer is intended herein to include any device that has a specialized processor as described above. For example, a computer may be a personal computer (“PC”), laptop computer, set top box, cell phone, smartphone, tablet device, smart wearable device, portable media player, video player, etc. The configurations provided for herein are discussed in the context of a mobile computing device, but may, alternatively, be implemented via other forms of computers.

It is understood that the apparatuses, systems, computer program products, and processes described herein may also be applied in other types of apparatuses, systems, computer program products, and processes. Those skilled in the art will appreciate that the various adaptations and modifications of the embodiments of the apparatuses described herein may be configured without departing from the scope and spirit of the present apparatuses, systems, computer program products, and processes. Therefore, it is to be understood that, within the scope of the appended claims, the present apparatuses, systems, computer program products, and processes may be practiced other than as specifically described herein.

I claim:

1. A mobile computing device comprising:
an image capture device configured to scan a QR code adhered to a medical linen;
a processor that generates a graphical user interface having a scanning menu, the scanning menu listing one or more user selectable indicia, each of the one or more user selectable indicia being selectable prior to the image capture device performing the scan of the QR code, each of the one or more selectable indicia being associated with a computer-implemented instruction to update a database via a network, the database storing a data structure corresponding to the medical linen, the one or more selectable indicia being selected from the group consisting of: a medical linen installation indicium and a medical linen disposal indicium;
a display device that displays the graphical user interface and the scanning menu within the graphical user interface;
a receiver that receives a user input, via the graphical user interface, of a selected indicium from the one or more user selectable indicia;
a memory device that stores data corresponding to the selected indicium, the memory device further storing the image of the QR code subsequent to storing the selected indicium; and
a transmitter that transmits the selected indicium and the QR code to the server.

2. The mobile computing device of claim 1, wherein the group further consists of: a laundered medical linen indicium.

3. The mobile computing device of claim 1, wherein the medical linen is a textile-based medical linen.

4. The mobile computing device of claim 1, wherein the medical linen is a disposable medical linen.

5. The mobile computing device of claim 1, wherein the medical linen is a curtain that separates a plurality of patient beds in a healthcare environment.

6. The mobile computing device of claim 1, wherein the receiver is further configured to receive, from the server, a laundering history of the medical linen, and the display device is further configured to display the laundering history.

7. The mobile computing device of claim 1, wherein the user input of the selected indicium activates a computer implemented instruction to scan the QR code.

8. The mobile computing device of claim 7, wherein the laundering history includes an installation date.

9. The mobile computing device of claim 7, wherein the laundering history includes a date of last laundering.

10. The mobile computing device of claim 7, wherein the laundering history includes a quantity of times the medical linen was previously laundered.

11. A computer program product comprising a non-transitory computer readable medium having a computer readable program stored thereon, wherein the computer readable program when executed on a computer causes the computer to:
scan, with an image capture device, a QR code adhered to a medical linen;
generate, with a processor, a graphical user interface having a scanning menu, the scanning menu listing one or more user selectable indicia, each of the one or more user selectable indicia being selectable prior to the image capture device performing the scan of the QR code, each of the one or more selectable indicia being associated with a computer-implemented instruction to update a database via a network, the database storing a data structure corresponding to the medical linen, the one or more selectable indicia being selected from the group consisting of: a medical linen installation indicium a medical linen disposal indicium;
displaying, with the display device, the graphical user interface and the scanning menu within the graphical user interface;
receive a user input, via the graphical user interface, of a selected indicium from the one or more user selectable indicia;
store, at a memory device, data corresponding to the selected indicium and the image of the QR code subsequent to storing the selected indicium; and
transmit, with a transmitter, the selected indicium and the QR code to the server.

12. The computer program product of claim 11, wherein the group further consists of: a laundered medical linen indicium.

13. The computer program product of claim 11, wherein the medical linen is a textile-based medical linen.

14. The computer program product of claim 11, wherein the medical linen is a disposable medical linen.

15. A system comprising:
a database that stores a data structure corresponding to a medical linen;
a processor that generates a graphical user interface having a scanning menu, the scanning menu listing one or more user selectable indicia, each of the one or more user selectable indicia being selectable prior to an image capture device of a mobile computing device performing a scan of a QR code adhered to a medical linen, each of the one or more selectable indicia being associated with a computer-implemented instruction to update the database via a network, the one or more selectable indicia being selected from the group consisting of: a medical linen installation indicium and a medical linen disposal indicium; and
a transmitter that sends the graphical user interface to the mobile computing device prior to a user selection being performed from the scanning menu.

16. The system of claim 15, further comprising a receiver that receives a selected indicium from the one or more user selectable indicia, the receiver further being configured to receive the QR code.

17. The system of claim 16, wherein the processor is further configured to decode the QR code to determine a medical linen identifier, query the database based upon the medical linen identifier, retrieve medical linen data associated with the medical linen from the data structure, perform the computer-implemented instruction on the medical linen data, and update the database with updated medical linen data.

18. The system of claim 17, wherein the transmitter is further configured to send the updated medical linen data to the mobile computing device, the mobile computing device displaying the updated medical linen data in the graphical user interface of the mobile computing device.

19. The system of claim 15, wherein the data structure stores a laundering history for the medical linen.

20. The system of claim 19, wherein the transmitter is further configured to send the laundering history to the mobile computing device, the mobile computing device displaying the laundering history in the graphical user interface of the mobile computing device.
